(19) United States
(12) Patent Application Publication
(10) Pub. No.: US 2010/0056898 A1
(43) Pub. Date: Mar. 4, 2010

(54) Objective Methods of Estimating Age of Animals and Carcasses

(76) Inventors: David R. McKenna, Benton, KS (US); Daniel L. Schaefer, Wichita, KS (US)

Correspondence Address: CARGILL, INCORPORATED, P.O. Box 5624, MINNEAPOLIS, MN, 55440-5624, US

(21) Appl. No.: 11/922,077

(22) PCT Filed: Jun. 13, 2006
(86) PCT No.: PCT/US2006/023084

§ 371 Date: Oct. 15, 2009

Related U.S. Application Data
(60) Provisional application No. 60/689,827, filed on Jun. 13, 2005.

(51) Int. Cl. A61B 5/055; A61B 8/00; A61B 6/00; G01N 23/06

(52) U.S. Cl. 600/411; 600/437; 600/473; 600/476; 378/62

(57) ABSTRACT

An age of an animal or animal carcass is estimated using a measurement or measurements of physical properties of the animal or carcass. A correlation between at least one physical property or characteristic and the animal's age is determined. A measurement of the at least one physical property or characteristic is made using non-invasive technology. An estimated age is then determined comparing the measured at least one property to the correlation of property and age.

### REFERENCE TO RELATED APPLICATIONS

This application claims priority to International Application Number PCT/US2006/023084 entitled, “OBJECTIVE METHODS OF ESTIMATING AGE OF ANIMALS AND CARCASSES” having an International Filing Date of Jun. 13, 2006, and which is herein incorporated by reference in its entirety and this application claims priority to U.S. Provisional Patent Application No. 60/689,827 filed on Jun 13, 2005, entitled “OBJECTIVE METHODS OF ESTIMATING AGE OF ANIMALS AND CARCASSES” and which is herein incorporated by reference in its entirety.

### FIELD OF INVENTION

This invention relates generally to methods for determining the age of animals, and more specifically to methods for measuring physical properties of animals to determine a likely age using non-invasive technologies.

### BACKGROUND

It is often times desirable or important for a packer to be able to determine the age of an animal at the time of slaughter. A particular need to determine the age of animals at slaughter exists in the beef industry. The emergence of bovine spongiform encephalopathy (BSE) has led to the need to differentiate animals that are older than 30 months of age at the time of slaughter. Further, for some export markets such as Japan, it is necessary to differentiate animals that are younger than 20 months of age at the time of slaughter. Currently, chronological age is estimated by subjective observations by trained personnel. Most commonly, a visual inspection of an animal's teeth is used to estimate that animal's age. For carcass grading purposes, the USDA Agricultural Marketing Service evaluates the amount of ossification in the thoracic, lumbar, and sacral vertebrae. Because of variations in biology from animal to animal, and because of the subjective nature of the observations, these methods are not as accurate as desired.

There is therefore a need for a method of estimating the age of an animal, or animal carcass, based on objective criteria.

### BRIEF SUMMARY OF THE INVENTION

According to one embodiment, the present invention provides a method for non-invasive determination of age of an animal by observing a physical characteristic using a non-invasive technique, and correlating that characteristic to age of the animal.

In one embodiment, the physical characteristic is a characteristic of a bone, and the observing is done using an imaging technology such as X-ray, magnetic resonance imaging, or ultrasound.

In another embodiment, the physical characteristic is a characteristic of connective tissue, and the observing is done using a spectroscopic technology such as near infra-red, infra-red, fluorescence, or Raman spectroscopy.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a flow chart illustrating a method of estimating the age of an animal based on observation of a physical characteristic in accordance with one embodiment;

FIG. 2 is a flow chart illustrating a method of estimating the age of an animal based observation of more than one physical characteristics in accordance with one embodiment;

FIG. 3 illustrates an arrangement for estimating the age of an animal using spectrophotometry in accordance with one embodiment;

FIG. 4 illustrates a partial cross-sectional view of an imaging cabinet in accordance with one embodiment;

FIG. 5 is a graph showing results of a case study using NIR technology to estimate whether cattle carcasses were older than 30 months of age;

FIG. 6 illustrates an arrangement for estimating the age of an animal using spectrophotometry in accordance with a further embodiment;

FIG. 7 illustrates an enlarged view of the calcanean tendon in a beef carcass as shown in the arrangement of FIG. 6;

FIG. 8 illustrates an arrangement of a carcass portion for an imaging cabinet in accordance with a further embodiment embodiment;

FIG. 9 illustrates an enlarged front view of the left carpus in a beef carcass as shown in the arrangement of FIG. 8;

FIG. 10 illustrates an enlarged side view of the left carpus in a beef carcass as shown in the arrangement of FIG. 8;

FIG. 11 illustrates a median cross section of a long bone of a younger animal; and

FIG. 12 illustrates a median cross section of a long bone of an older animal.

### DESCRIPTION OF PREFERRED EMBODIMENTS

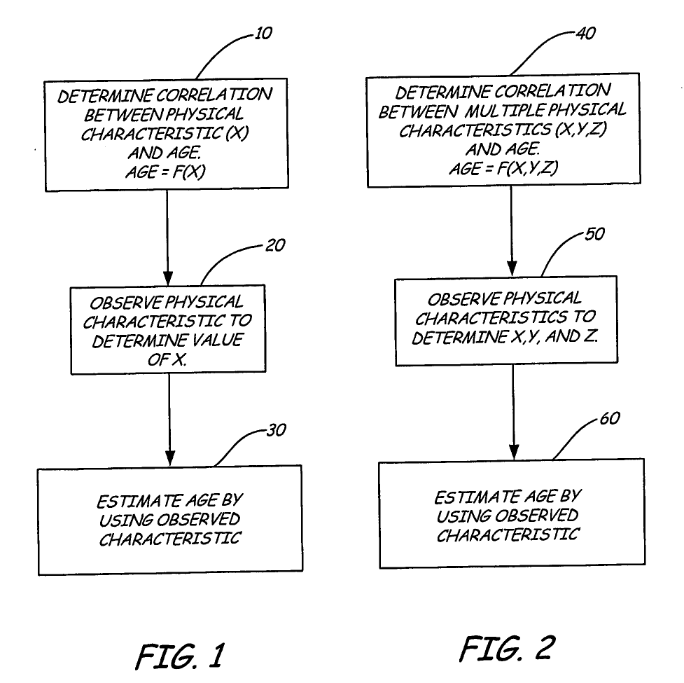

Methods for determining the age of an animal prior to, or shortly after, slaughter are provided. While the drawings and embodiments discussed relate primarily to cattle and beef carcasses, the methods may be used for with any meat-carcasses (for example pork, lamb, veal, cow, and bull), poultry (for example, turkey and chicken) or fish from any source. The present invention may be applied to live animals of any age. That is, the present invention may be applicable to an animal at any stage of its life (i.e., birth to death—whatever age that may be). FIG. 1 graphically illustrates a generalized process for determining the age of an animal or animal carcass. According to the embodiment of FIG. 1, the first step 10 is to determine a relationship between a physical characteristic of the animal or animal carcass and the age of the animal. By way of example only, where an animal's bone density decreases in a predictable manner over time, it is possible to estimate the age of the animal by measuring bone density. This relationship may be determined by measuring the physical characteristic in animals of known age, and then plotting the physical characteristic as a function of age. A regression analysis may be performed to determine a best fit curve that describes the relationship. This can be used to generate an equation for determining age as a function of characteristic being measured (i.e., age=f(x), where x is measurement of characteristic). Thus, age is determined as a function of that physical property.

Alternatively, a graphical method may be used. For example, specimens of known age may be measured for two different properties and plotted on a graph with a first axis corresponding to a first property and a second axis corresponding to a second property. A pattern may be apparent that permits an estimation of an animals age depending upon where on the graph the two measures fall.

Generally, any suitable manner of generating an equation or representative graph may be used for correlating at least one property with age. Further, any suitable property varying with age may be used. Those of skill in the art will be aware of techniques for performing this regression analysis, and for determining samples sizes necessary to develop an accurate relationship. In some instances, the relationship may have been determined by prior research conducted by third parties.

The second step 20 of FIG. 1 is to observe the physical property in an animal or carcass of unknown age. Preferably this observation takes the form of an objective measurement. The observation may comprise determining a length, or ratio of lengths, of an at least one portion of the animal or carcass. The observation may involve the use of technologies such as spectroscopic devices or imaging technologies, as will be described in more detail below.

Once the physical property has been observed in the animal or carcass of unknown age, the final step 30 is to determine the age of the animal or carcass based on the observed property. This determination may be done by inserting the property value into the equation described in the first step 10 to determine and estimated age. Alternatively, the determination may be done by charting the value of the observed characteristic on a graph of such characteristic versus age.

FIG. 2 illustrates an embodiment where more than one physical characteristic is used to estimate an age of an animal or carcass. The first step 40 in the process may include determining a relationship between multiple physical properties of an animal or carcass and age of that animal or carcass (i.e., age=f(x,y,z), where x, y, and z are all different characteristics). For example, the bone density of a specified bone and the elasticity of a specified tendon may be measured in several animals of known ages. A best fit curve may then be determined for age as a function of elasticity and density (i.e., age=f(e,d), where e is elasticity and d is density) through known regression analysis techniques.

The second step 50 is to measure the physical characteristics used to estimate an age of an animal or carcass of the first step 40 in animals or carcasses of unknown age. Measurement, or observation, may be done using imaging technology, spectroscopic technology, or other suitable technique.

The final step 60 is to correlate the measured or observed physical characteristics with age to determine the age of the animal or carcass. In one embodiment, such correlation done by inserting the observed values into the best fit equation determined through the regression analysis to arrive at an estimated age of the animal or carcass. In another embodiment, such correlation is done by charting the values of the measured characteristics on a graph of such characteristics versus age.

In alternative embodiment using a plurality of observed or measured characteristics, the age values determined by individual characteristic regression analysis may be averaged to determine an average estimated age for each value. According to this embodiment, best fit curves are determined for age as a function of each individual variable physical characteristic, as described with respect to the method of FIG. 1. The results of each analysis are then averaged to arrive at an average estimated age. A first estimated age may be determined using the best fit curve for the age of the animal with respect to a first property, and a second estimated age may be determined by using the best fit curve for a second physical property. An average estimated age may then be determined by averaging the first and second estimated ages determined by each of the two physical properties. Numerous properties and best fit curves may be determined and averaged to determine an average estimated age. While averaging a plurality of estimated ages is discussed with respect to determining age based on a best fit curve of an observed characteristic versus age, other means of determining an estimated age may be used. Regardless of manner of determining estimated age, if more than one characteristic is used such that more than one estimated age is determined, the estimated ages may be averaged to determine an average estimated age.

Those of skill in the art will be aware of numerous physical properties and numerous observation techniques that may be utilized in the above-described fashion to determine an estimated age for animals and animal carcasses. Specific example embodiments are described in more detail below.

### Use of Spectrophotometry to Estimate Age

As an animal ages, physical properties of its connective tissue change. For example, as a mammal ages, the amount of cross-linking in its collagen increases. This cross-linking is a result of a glycosylation reaction between adjacent strands of collagen. This glycosylation reaction occurs at a predictable rate. The level of cross-linking can be measured directly using near infrared (NIR) spectrophotometry. NIR measures shifts in the spectral reflectance at certain wavelengths. In the case of collagen, the range of emphasis is 1600 to 1700 nm wavelengths. This range has been established as measuring many of the collagen interaction bonds, although other ranges along the spectra may include important information and may be used. In addition to NIR technology, infra-red (IR), fluorescence, and Raman spectroscopy may be used to estimate the level of cross-linking in connective tissue.

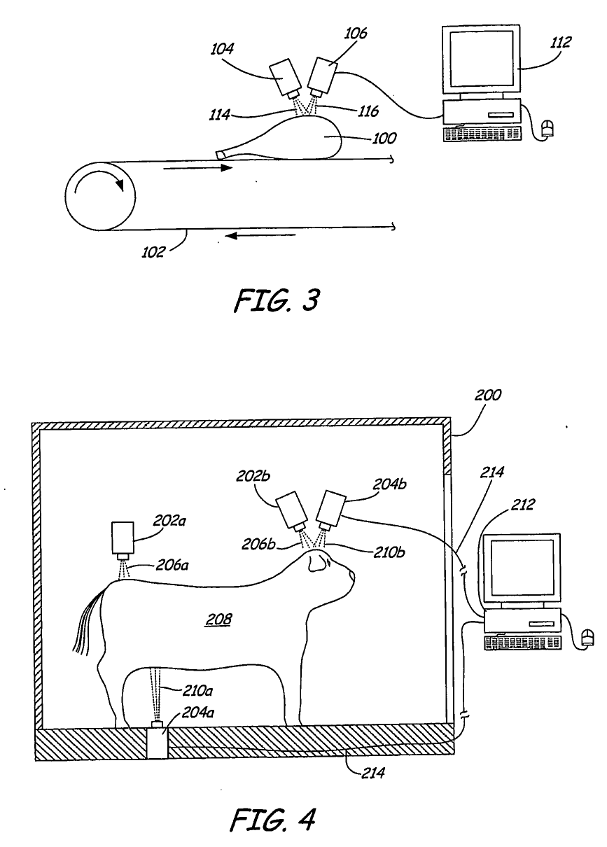

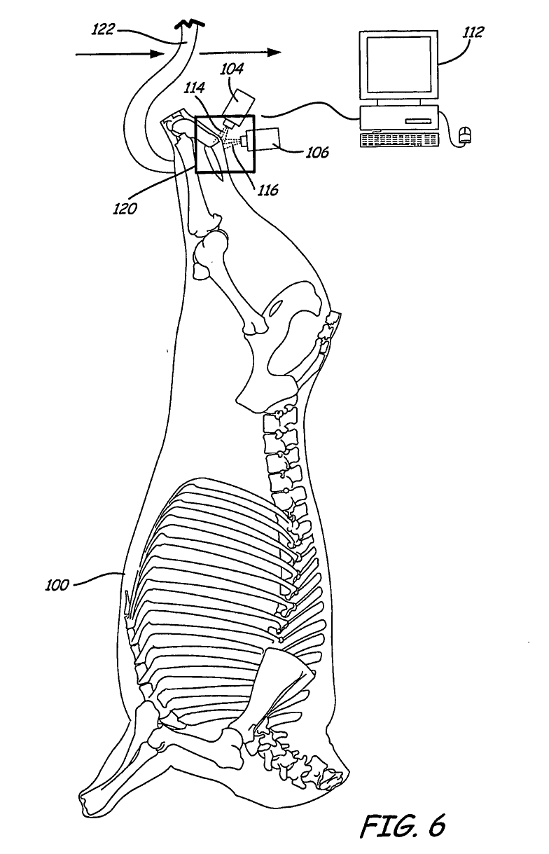

An arrangement for estimating the age of an animal carcass by measuring the level of cross-linking is illustrated in FIG. 3. An alternative arrangement for estimating the age of an animal carcass by measuring the level of cross-linking is illustrated in FIG. 6. In the embodiment of FIG. 3, a portion of an animal carcass 100 rests on a conveyor belt 102. In contrast, in the embodiment of FIG. 6 an animal carcass is vertically suspended by its common calcanean tendon (may be referred to in the art as the “gambrel” tendon or the “Achilles” tendon) 120 using a hook 122. Thus, the animal carcass portion 100 may be moved by a conveyor 102, by traveling hook 122, manually, or by other suitable mechanisms known to those in the art. In alternative embodiments, the animal carcass portion 100 may be stationary.

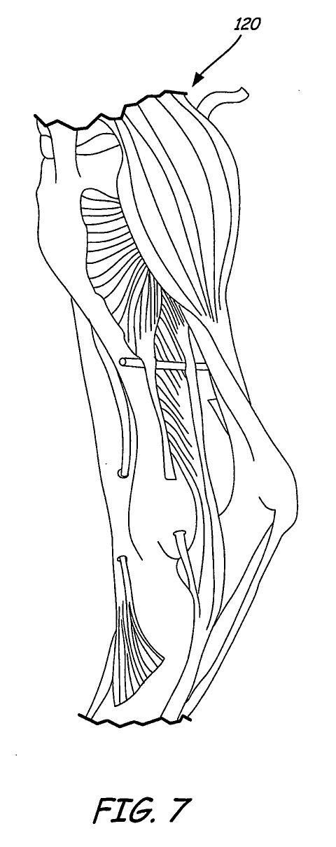

Regardless of orientation of the animal carcass portion 100, an emitter 104 and a receiver 106 are mounted in an operable position near the travel path of the carcass, for arrangements involving travel of the carcass, or near the carcass, for stationary arrangements. The emitter 104 emits a signal 114 which is reflected off of the animal carcass 100 as reflected signal 116, which is received by receiver 106. In practice, the emitter 104 and receiver 106 may be a single spectroscopy device such as an NIR, IR, fluorescence, or Raman spectrometer. According to one embodiment, the emitter 104 and receiver 106 are mounted near a hind-leg transfer station to measure physical properties of a calcanean tendon in a beef carcass. FIG. 7 illustrates an enlarged view of the calcanean tendon 120 of a beef carcass. Other embodiments include measuring external connective tissue on the shoulder, the internal portion of the hide, or other characteristic of the animal carcass portion.

The receiver 106 translates the reflected signal 116 from the animal carcass portion 100 into data that is transferred to a computer central processing unit (CPU) 108, for example by wire 110. Other mechanisms such as RF or IR signals may be used to transmit the data from the receiver 106 to the CPU 108. The CPU 108 may write the data to a hard drive, or other storage device. In one embodiment, the CPU 108 is loaded with software to permit the CPU 108 to compute an estimated age of the animal carcass portion based on a known or determined relationship between the level of cross-linking and the age of the animal. In alternative embodiments, the estimated age is determined manually, for example by correlating the observed or measured value with a graph of the value versus age.

A monitor, or display screen 112 may be provided with the CPU 108 to display information. The monitor 112 may display the readings of specific cross-linking levels within the animal carcass portions 100 as well as the estimated age for those portions 100. The CPU 108 may be connected to a network to transmit information related to an animal carcass portion 100 to additional computers. A warning signal may be displayed on the monitor 112 in case of erroneous or unrecognized readings from the receiver 106. Additionally the CPU 108 may provide a signal, for example a visual or audible signal, in case the estimated age of an animal carcass portion 100 is older than a desired age.

It should be appreciated that, in arrangements where the carcass portion is moving, the travel of the animal carcass portion 100 may be halted at least momentarily to ensure an accurate reading of the reflected radiation may be obtained by the receiver 106. In alternative embodiments, such halting may not be necessary. Further, in some situations, it may be necessary or desirable to manually arrange the animal carcass portion 100 into a desired position and orientation in order to best get an accurate reading. It may also be desirable to take several measurements of the level of cross-linking at various points on the animal carcass portion 100 to determine an average level of cross-linking within that animal carcass portion 100.

Any other suitable method for measuring the amount of cross-linking in connective tissue may alternatively be used. Thus, other indirect methods may be used to measure the amount of cross-linking in connective tissue. For example, the thermal transition temperature is the temperature at which collagen is converted into gelatin. The thermal transition temperature of collagen increases as the amount of cross-linking increases. Collagen from older animals has accumulated more cross-links and, thus, will have an elevated thermal transition temperature as compared to collagen from younger animals. Those of skill in the art will be aware of various methods for measuring the transition temperature of collagen specimens.

Similarly, as the cross-linking within connective tissue increases, the elasticity of that connective tissue decreases. Therefore, an alternative method of estimating the age an animal carcass is to test the elasticity of a portion of the carcass and compare it to a predetermined regression curve or expected age as a function of elasticity. The gambrel tendon on beef hind quarter is specific example of connective tissue that may be used for this type of measurement. Elasticity may be measured in a number of ways, including, without limitation, compression tests, deformation tests, tensile strength tests, or a combination of such tests. Two commercially available products that might be used in conducting such tests are a Universal Materials Testing Machine, by Instron (Norwood, Mass.) and TA.TX2 by Texture Technologies (Scarsdale, N.Y.). Other suitable methods for measuring elasticity known to those skill in the art may also be used.

Additionally, or alternatively, other physical features that change with age may be measured in the connective tissue of animals using spectroscopic devices. Such features include collagen form, isometric tension, thermal transition temperature. The relationships and ratios that exist between such measures may be used to estimate the age of an animal or carcass.

### Use of Imaging Technologies to Estimate Age

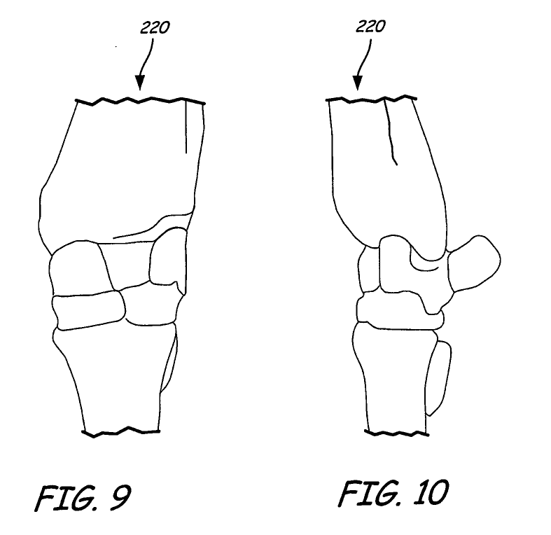

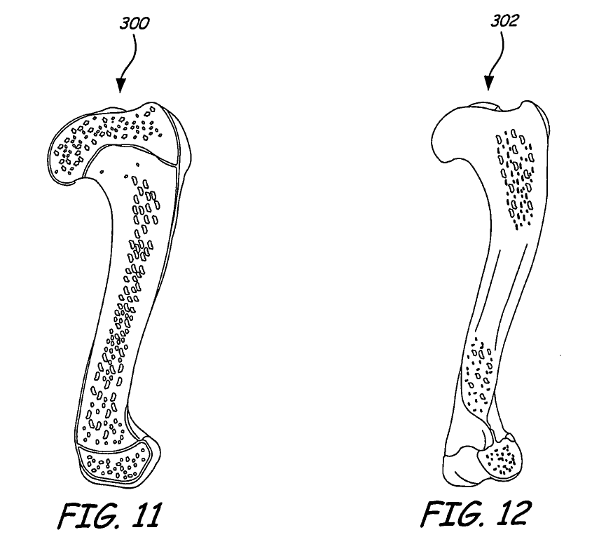

Bone is a dynamic tissue with physical properties that undergo changes as an animal ages. These physical properties include, for example, bone density, bone length and diameter, internal cavity characteristics such as porosity, hollowness and the like, mineral deposit composition, and growth plate properties. For example, FIGS. 11 and 12 illustrate comparative bone density of a long bone 300 of a younger animal (FIG. 11) versus the bone density of a long bone 302 of an older animal (FIG. 12). As shown, the density of the bone increases with age. FIGS. 9 and 10 illustrate alternative views of a left carpus of a cattle carcass. As shown, the carpus includes plurality of bones and growth plates. Any of these bones or growth plates may be suitable for measurement or observation. Imaging technologies such as X-ray, ultrasound, and magnetic resonance can be used to measure these properties. Imaging technologies can also be used to observe tooth development prior to eruption.

As discussed above, a first step for estimating age is to determine a relationship between one or more physical features that can be observed through imaging technologies and the age of the animal. This may be done by observing samples of animals of known ages, and then conducting regression analyses. The relationship may be based on observation of a single property (i.e., age=f(x), where x is a measure of a physical property) (see FIG. 1), or, may be based on observations of more than one property (i.e., age=f(x,y,z), where x, y, and z are all measures of different physical properties) (see FIG. 2).

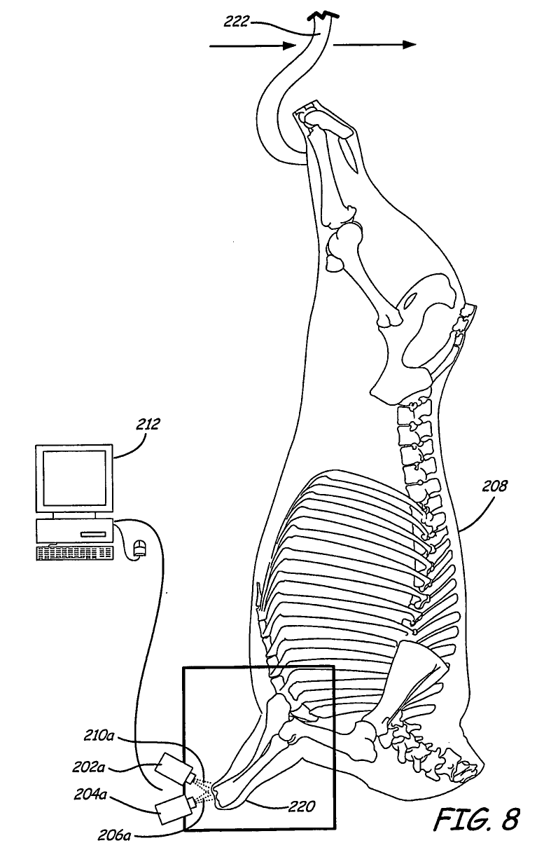

FIGS. 4 and 8 illustrate embodiments of an arrangement for observing physical properties using imaging technologies. FIG. 4 illustrates an imaging cabinet 200 with a carcass portion provided in a first orientation therein. FIG. 8 illustrates an alternative orientation for a carcass portion within an imaging cabinet. In the embodiment of FIG. 4, the carcass 208 is placed substantially in an upright position in the imaging cabinet 200. In the embodiment of FIG. 8, the animal carcass 208 is vertically suspended by its common calcanean tendon using a hook 222. While a specific embodiment of an imaging cabinet is shown and discussed, alternative means such as a handheld device or an operator applied stationary unit may be used.

According to the embodiment of FIG. 4, an imaging cabinet 200 is provided that includes a pair of emitters 202a and 202b and a pair of receivers 204a and 204b. In the embodiment of FIG. 8, a single emitter 202a and receiver 204a is provided. The emitters 202a and 202b emit imaging signals 206a and 206b respectively. An animal or carcass 208 is placed, or herded, into a desired position within the cabinet 200. As may be appreciated, the embodiment of FIG. 4 is well suited for live animals whereas the embodiment of FIG. 8 is suited for animal carcasses. As an imaging signal 206a, 206b encounters the animal 208, it is modified into a refracted or reflected signal 210a, 210b that represents the physical property Demg measured, 11ie reiracteα signal 210a, 210b is received by a corresponding receiver 204a, 204b, which converts the signal into data that can be transferred to a computer 212, for example by data cables 214. The computer 212 may be provided with software that performs the necessary calculations to estimate the age of the animal 208, based on the predetermined relationship between the physical characteristics and the animal's age. In alternative embodiments, the estimated age is determined manually, for example by correlating the observed or measured value with a graph of the value versus age.

Each of the pairs of emitters 202a, 202b and receivers 204a, 204b correspond to a specific type of imaging technology. For example emitter 202a and receiver 204a might be an X-ray, while 202b and 204b might be formed by a magnetic resonance imaging machine. Any combination of technologies may be used. In some embodiments, the same technology may be used for each of the emitters 202a, 202b and receivers 204a, 204b. In some embodiments, only a single emitter 202a and receiver 204a is used.

In some embodiments, a user may provide input to the computer 212 to specify the data to be used. For example, where the first emitter 202a and receiver pair 204a are an X- ray and the variable being measured is the length of a portion of a bone, the user may provide input to the computer 212 as to what portion of the bone to measure.

It should be appreciated that the cabinet 200 may include any number of emitter and receiver pairs comprising any combination of imaging technologies. Alternatively, each imaging technology measurement may be taken at separate cabinets 200 or at other locations in the process. Separate computers 212 may be attached to each receiver. The measurements need not be taken on the same portion of the carcass. For example, if the two factors being correlated are length of a portion of a leg bone and a density of a jaw bone, the measurements may be made after the animal has been slaughtered and separated into parts.

It should further be appreciated that spectroscopic and imaging technology methods could be combined in estimating an animal's age. For example, a first physical characteristic could be measured by spectroscopic means and a second physical characteristic by imaging technology means. A correlation based on the two factors could be determined using regression analysis, and the age of animals could then be estimated based on measurements of the two factors.

### Case Study

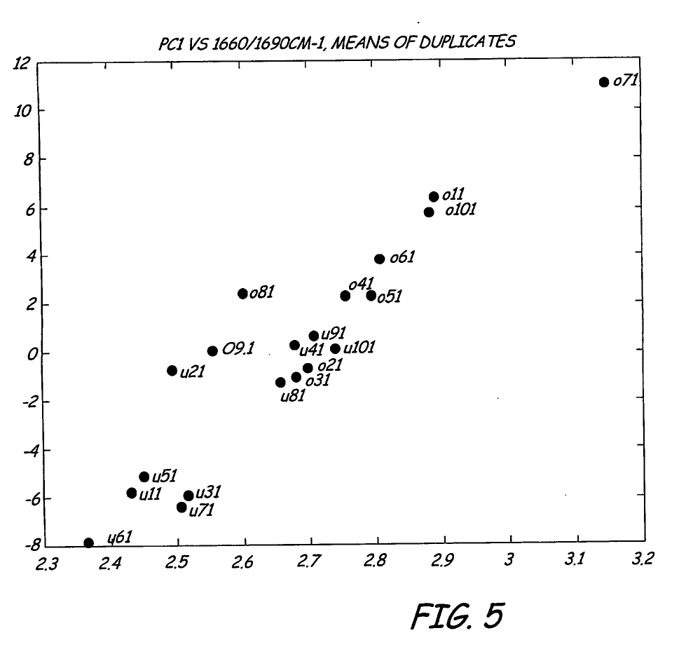

A case study was performed using NIR technology to differentiate collagen cross-links in beef tendons. According to the study, a shift in the spectral profile of beef tendons was measured. FIG. 5 shows the results of the study. The horizontal axis reflects a Principal Component (PC) analysis of the entire spectra (400 to 2500 ran) (Standard regression analysis technique that can establish relationship between x's (horizontal axis) that can help predict y (vertical axis); There are no true units to principal components) whereas the vertical axis corresponds to a ratio of the reflectance at 1660 nm:1690 nm. That ratio is one that is commonly used to investigate bonds/properties in collagen. Numeric values were randomly assigned to samples just to maintain sample identity, but there is no correlation between the numbers in each group. Data points labeled “O” represent animals older than 30 months of age, and data points labeled “U” represent animals younger than 30 months of age. As can be seen, animals older than 30 months of age tend to be located in the upper right quadrant, whereas animals younger than 30 months of age tend to be located in the lower left quadrant. This method therefore represents a graphical method of estimating whether a an animal is older or younger than 30 months of age.

Although the invention has been described with reference to preferred embodiments, persons skilled in the art will recognize that changes may be made in form and detail without departing from the spirit and scope of the invention.

1. A method for non-invasive determination of age of an animal or carcass comprising:
observing a bone characteristic using a non-invasive technique;
correlating the bone characteristic to age of the animal.

2. The method of claim 1, wherein the non-invasive technique comprises taking an x-ray.

3. The method of claim 1, wherein the non-invasive technique comprises performing an ultrasound.

4. The method of claim 1, wherein the non-invasive technique comprises magnetic resonance imaging.

5. The method of claim 1, wherein the bone characteristic is growth of the plates of bones.

6. The method of claim 1, wherein the bone characteristic is calcification of bones.

7. The method of claim 1, wherein the bone characteristic is hollowness of bones.

8. The method of claim 1, wherein the bone characteristic is density of bones.

9. A method for non-invasive determination of age of an animal comprising:
observing the amount of collagen in connective tissue of muscles of the animal using non-invasive near infrared (NIR) technology; and
correlating the amount of collagen in connective tissue to age of the animal.

10. A method of estimating the age of an animal comprising:
determining a correlation between a measurement of a physical characteristic and an age of a type of animal; measuring said physical characteristic in a specimen of said type of animal; and
determining an estimated age for said specimen by using said measured physical characteristic of said specimen in said determined correlation.

11. The method of claim 10, wherein said measuring step is performed using spectroscopic techniques.

12. The method of claim 11, wherein said spectroscopic technique utilizes near infra-red spectrophotometry.

13. The method of claim 11, wherein said spectroscopic technique utilizes infrared spectrophotometry.

14. The method of claim 11, wherein said spectroscopic technique utilizes fluorescence spectrophotometry.

15. The method of claim 11, wherein said spectroscopic technique utilizes Raman spectrophotometry.

16. The method of claim 11, wherein said physical characteristic is a level of cross-linking in connective tissue.

17. The method of claim 10, wherein said measuring step is performed using imaging technology.

18. The method of claim 17, wherein said imaging technology is an X-ray.

19. The method of claim 17, wherein said imaging technology is magnetic resonance imaging.

20. The method of claim 17, wherein said imaging technology is ultrasound.

21. A method of estimating the age of an animal comprising:
determining a correlation between a measurement of a first physical characteristic and a measurement of a second physical characteristic and an age of a type of animal having the first and second physical characteristics; measuring said first physical characteristic in a specimen of said type of animal; measuring said second physical characteristic in said specimen; determining an estimated age for said specimen by using said measured first and second physical characteristic of said specimen in said determined correlation.
